(19) United States
(12) Patent Application Publication
(10) Pub. No.: US 2023/0127872 A1
(43) Pub. Date: Apr. 27, 2023

(54) METHOD FOR ENHANCING CHARACTERISTICS OF PLANT

(71) Applicants: FREEZE-THAW AWAKENING TECHNOLOGY CO., LTD., Okayama (JP); AGRI INTERNATIONAL INC., Tokyo (JP); AGRICULTURAL CORPORATION D&T FARM INC., Okayama (JP)

(72) Inventor: Setsuzo TANAKA, Okayama (JP)

(21) Appl. No.: 17/914,205

(22) PCT Filed: Mar. 26, 2021
(86) PCT No.: PCT/JP2021/013130

§ 371 Date: Sep. 23, 2022

(30) Foreign Application Priority Data
Mar. 26, 2020 (JP) 2020-056248
Sep. 15, 2020 (JP) 2020-154584

(51) Int. Cl. A01H 1/00; A01H 3/04

(52) U.S. Cl. CPC A01H 1/12; A01H 3/04

(57) ABSTRACT

The present invention addresses the problem of providing a novel way of enhancing the characteristics of a plant without using genetic recombination. The present invention involves freezing plant tissue during a freezing step and then obtaining a liquid extract from the frozen plant tissue.

### TECHNICAL FIELD

The present invention relates to a method for enhancing characteristics of a plant without using gene manipulation.

### BACKGROUND ART

Since ancient times, human beings have produced a plant having advantageous properties by a breeding technique. The conventional breeding method requires a long time to fix the certain characteristics, however, with the advent of a generation acceleration technique, the time required for fixing the certain characteristics can be shortened. However, even with the generation acceleration technique, there has been a problem that it takes several years to fix the certain characteristics. Therefore, biotechnology such as anther culture that does not require any fixing work has been developed.

Further, as the method for producing a plant having advantageous characteristics, a gene recombination technique is known. By the gene recombination technique, herbicide-tolerant crops, pest-resistant crops, disease-resistant crops, and crops with increased preservation have been produced.

On the other hand, there has been proposed a method for inducing mutation and enhancing characteristics of a plant by performing a certain treatment. For example, in Patent Literature 1, a breeding method for imparting cold tolerance, including a step of performing gamma irradiation and chromosome doubling treatment has been disclosed.

Further, a method for controlling characteristics of a plant without changing the gene sequence has been devised. For example, in Patent Literature 2, a method for controlling the flowering time in the next generation of a plant by applying a stress treatment of salt stress, poor sunshine stress, strong light stress, drought stress, over-humidity stress, high-temperature stress, low-temperature stress, nutrient stress, heavy metal stress, disease stress, oxygen deficiency stress, ozone stress, CO 2 stress, strong wind stress, or the like due to the cultivation environment in the vegetative growth time of a plant has been disclosed.

By the way, most of the areas in Japan belong to the temperate zone, and Hokkaido and Tohoku region belong to the subarctic zone (cool-temperate zone). Therefore, crops that are not suitable for the cultivation in a climate in Japan, such as those cultivated in from the subtropical zone area to the tropical zone area, are in a situation of being dependent on the import.

As an epoch-making technology to solve this problem, a technology called “freezing and thawing awakening method” (Patent Document 3) was developed by the inventor, and has achieved a large number of very good results so far. For example, domestically produced pesticide-free bananas have been produced by applying the freezing and thawing awakening method, and those produced in Okayama Prefecture are sold under the name “Monge Banana” (registered trademark).

The freeze-thaw awakening method is a method of cultivating frozen and thawed plant tissue to enhance the plant's characteristics, specifically, environmental adaptation characteristics such as growth rate, cold tolerance, high temperature adaptation, highland and lowland adaptation characteristics, fruit and seed quantity and size, sweetness, pest and disease resistance, and drought tolerance. The freeze-thaw awakening method is not limited to plants of a certain family, genus, or species, but can be applied to all plants. To date, more than 230 varieties have been successfully cultivated.

### CITATION LIST

### Patent Literature

Patent Literature 1 JP2006-25632
Patent Literature 2 JP2016-182094
Patent Literature 3 JP2018-183112.

### SUMMARY OF INVENTION

### Technical Problem

The problem to be solved is to provide a new technology to enhance plant characteristics without genetic modification.

### Solution to Problem

In the process of researching further development of the freeze-thaw awakening method, the inventor discovered that by immersing other plant tissues in the extraction liquid of one plant tissue that has undergone the freezing step, the same effect can be given as if the freeze-thaw awakening method were applied to said other plant tissue. Specifically, the present invention provides the following.

[1] A method for producing an extraction liquid, comprising

a freezing step for freezing plant tissue and

an extraction step for obtaining an extraction liquid from the plant tissue that has undergone the freezing step.

[2] The method for producing an extraction liquid according to [1], wherein the extraction liquid for enhancing the characteristics of a plant.

[3] The method for producing an extraction liquid according to [2], wherein the characteristics of a plant are one or more of the following:

plant growth characteristics, cold tolerance, high temperature adaptation, germination rate, growth uniformity, degree of rooting, fertility, and drought tolerance.

[4] The method for producing an extraction liquid according to any one of [1] to [3], wherein comprising a selection step for selecting living plant tissue from the frozen plant tissue, and

the selection step is performed between the freezing step and extraction step.

[5] The method for producing an extraction liquid according to any one of [1] to [4], wherein in the freezing step, the plant tissue is frozen to −20° C. or lower over 100 days while lowering the temperature at a rate of 0.8° C./day or less.

[6] The method for producing an extraction liquid according to any one of [1] to [5], wherein the plant tissue is frozen while immersed in a sugar solution in the freezing step.

[7] The method for producing an extraction liquid according to [6], wherein the sugar is trehalose.

[8] The method for producing an extraction liquid according to [4], wherein in the selection step, a fermentation treatment is performed on the plant tissue that has undergone the freezing step.

[9] The method for producing an extraction liquid according to [8], wherein the fermentation treatment is performed by leaving the plant tissue that has undergone the freezing step in the open air.

[10] The method for producing an extraction liquid according to [9], wherein the leaving is performed at 0° C. to 40° C.

[11] The method for producing an extraction liquid according to any one of [8] to [10], wherein in the selection step, dead and living plant tissues are separated after the fermentation treatment.

[12] The method for producing an extraction liquid according to [11], wherein the separation treatment is performed by washing the fermented plant tissue.

[13] The method for producing an extraction liquid according to [12], wherein the washing is a water rinse.

[14] The method for producing an extraction liquid according to any one of [1] to [13], wherein in the extraction step, a living plant tissue is subjected to a crushing treatment.

[15] The method for producing an extraction liquid according to [14], wherein the crushing treatment is a grinding treatment.

[16] The method for producing an extraction liquid according to [15], wherein the grinding treatment is performed over a period of tens of seconds to several hours.

[17] The extraction liquid produced by the producing method according to any one of [1] to [16].

[18] The extraction liquid according to [17], wherein the extraction liquid is for enhancing plant characteristics.

[19] The extraction liquid according to [18], wherein the extraction liquid contains sugars or sugar alcohols.

[20] The extraction liquid according to [19], wherein the sugar or sugar alcohol is sucralose and/or trehalose.

[21] The extraction liquid according to any one of

[17] to [20], wherein the extraction liquid is diluted.

[22] The extract dried product produced by drying the extraction liquid according to any one of [17] to [21].

[23] A method of enhancing characteristics of a plant tissue, comprising:

a soaking step in which plant tissue from which the characteristics of the plant are to be enhanced is soaked in the extraction liquid,

wherein said extraction liquid is

the extraction liquid according to any one of [17] to

[21], or

the extraction liquid produced by dissolving the extract dried product according to [22].

[24] The method of enhancing characteristics of a plant tissue according to [23], wherein the plant tissue from which the characteristics of the plant are to be enhanced is dried prior to the soaking step.

[25] The method of enhancing characteristics of a plant tissue according to [24], wherein a soaking time in the soaking step is from 1 to 100 hours.

[26] A method for producing plant tissue with enhanced plant characteristics comprising applying the method of enhancing characteristics of a plant tissue according to any one of [23] to [25].

[27] A plant tissue with enhanced plant characteristics produced by applying the method according to

[26].

[28] A method for producing a plant with enhanced plant characteristics, comprising the step of growing the plant tissue according to [27].

[29] A method for enhancing plant characteristics, comprising a spraying step for spraying the extraction liquid on the plants for which the plant characteristics are to be enhanced,

wherein said extraction liquid is

the extraction liquid according to any one of [17] to

[21], or

the extraction liquid produced by dissolving the extract dried product according to [22].

[30] A method for producing a plant with enhanced plant characteristics, comprising applying the method for enhancing plant characteristics according to [29].

[31] A plant with enhanced plant characteristics produced by the method for producing according to [28] or [30].

[32] The plant according to [31], wherein the plant characteristics are one or more of the following:

plant growth characteristics, cold tolerance, high temperature adaptation, germination rate, growth uniformity, degree of rooting, fertility, and drought tolerance.

[33] A plant tissue obtained from the plant according to [32], and used as a scion for grafting.

[34] A plant grafted with the plant tissue according to [33] as a scion.

[35] A method for producing fruits or seeds of said plant comprising cultivating the plant according to [32] or [34].

[36] Fruits or seeds produced by the method according to [35].

[37] A method for searching for genes involved in enhancing plant characteristics, comprising;

A step of treating plants by the method according to any one of [23] to [25] and [29], and
(i) a step for identifying genes that are highly expressed in the treated plants as compared to plants that have not received said treatment, and/or
(ii) a step for identifying genes that are expressed at lower levels in the treated plants as compared to plants that have not received said treatment.

[38] A method for screening for enhancing factors of plant characteristics, comprising

using the following (i) or (ii) genes as screening indicator;
(i) A gene that is highly expressed in treated plants compared to plants that have not been treated by the method of any one of [23] to [25] and [29] and/or
(ii) A gene that is lower expressed in treated plants compared to plants that have not been treated by the method according to any one of [23] to [25] and [29];

screening the substance as an enhancing factor for a plant characteristic when the expression level of the gene described in (i) in plants treated with the test substance is higher than the expression level of the gene in plants not treated with the test substance and/or

screening the substance as an enhancing factor for a plant characteristic when the expression level of the gene described in (ii) in plants treated with the test substance is lower than the expression level of the gene in plants not treated with the test substance.

[39] A method for analyzing the extraction liquid comprising

preparing an extraction liquid produced by the method according to any one of [1] to [21] as an analysis object,

preparing, as a comparison object, an extract solution extracted from plant tissue that has not undergone the freezing process described above.

comparing and analyzing the extraction liquid of the analysis object and the extraction liquid of the comparison object, and

identifying a component wherein the component is

a component contained in the extraction liquid of the analysis object, but not in the extraction liquid of the comparison object and/or

a component contained more or less in the extraction liquid of the analysis object compared to the extraction liquid of the comparison object.

[40] A method for searching an enhancing factor for a plant characteristic comprising

preparing an extraction liquid produced by the method according to any one of [1] to [21] as an analysis object,

preparing, as a comparison object, an extract solution extracted from plant tissue that has not undergone the freezing process described above.

comparing and analyzing the extraction liquid of the analysis object and the extraction liquid of the comparison object, and

identifying a component wherein the component is

a component contained in the extraction liquid of the analysis object, but not in the extraction liquid of the comparison object and/or

a component contained more in the extraction liquid of the analysis object compared to the extraction liquid of the comparison object.

[41] The method for searching according to [40], comprising

a soaking step in which plant tissues or plant cells are soaked in a solution containing one or more components identified by the identifying, and

a determination step in which, if the plant characteristics are enhanced in the plant tissue or plant cells that have undergone the soaking step, the component is determined to be an enhancing factor for the plant characteristics.

[42] The method for searching according to [41], wherein in the determination step, the plant characteristics of plants generated from plant tissue or plant cells that have undergone the soaking step are observed, and if an enhancement of the plant characteristics is observed compared to plants generated from plant tissue or plant cells that have not undergone the soaking step, the component is determined to be an enhancing factor for the plant characteristics.

[43] The method for searching according to [41] or [42], wherein the determination step comprises

analyzing the expression level of the gene identified by the method according to [37] in the plant tissue or plant cells that have undergone the soaking step, and

determining that the component is the enhancing factor for the plant characteristics, when an increase in the expression level of the gene identified in (i) above is observed, and/or when a decrease in the expression level of the gene identified in (ii) above is observed.

[44] A method for producing liquid for enhancing plant characteristics, comprising a step for adding to an aqueous medium a component that has been determined to be an enhancing factor for the plant characteristics by the method for searching according to any one of [41] to [43].

[45] The method for producing according to [44], wherein the component added to said aqueous medium is obtained by extraction from plant tissue or by artificial synthesis.

[46] Solution comprising the component that has been determined to be an enhancing factor for the plant characteristics by the method for searching according to any one of [41] to [43].

[47] A method for enhancing characteristics of plant tissue, comprising a soaking step in which plant tissue from which plant characteristics are to be enhanced is soaked in a solution produced by the method according to [44] or [45].

[48] A method for enhancing plant characteristics, comprising a spraying step in which the solution produced by the method according to [44] or [45] is sprayed on plants for which the plant characteristics are to be enhanced.

According to the invention, plants with enhanced characteristics can be obtained without the need for multi-year breeding or genetic modification methods. In addition, in the freeze-thaw awakening method, individual plant tissues must undergo a prescribed freezing and thawing process, but in the present invention, once plant tissues with enhanced characteristics are obtained by the freeze-thaw awakening method, the time-consuming process of freezing and thawing is not necessary. Therefore, the advantage is that the superior characteristics can be imparted to a large amount of different plant tissues at once, quickly and cost-effectively, with a simple operation of simply obtaining an extraction liquid from the plant tissue and immersing it in it. This will further accelerate the speed of propagation of the technology of enhancing plant characteristics by the freeze-thaw awakening method, and will allow it to spread more and more widely to various plants and regions of the world.

Grains such as rice, wheat, wheat and soybeans are consumed in large quantities as staple food or feed, and are therefore grown in large quantities on huge acreage. In other words, the cultivation of grains requires a huge amount of seeds in order to supply a sufficient quantity of crops to meet demand.

Here, the method for enhancing characteristics has the advantage of imparting superior characteristics to large quantities of plant tissue at a time. Therefore, the method for enhancing characteristics of the present invention can be used very suitably for grain seeds that need to be planted in huge quantities.

### BRIEF DESCRIPTION OF DRAWINGS

FIG. 1: A photograph showing the results of Test Example 1. On the right is the wheat in the example and on the left is the wheat in the comparison.

FIG. 2: This photo shows the results of Test Example 1. The wheat planted in the right row is the example wheat and the wheat planted in the left row is the comparison wheat.

FIG. 3: This photo shows the results of Test Example 2. The wheat planted in the upper row is the example wheat and the wheat planted in the lower row is the comparison wheat.

FIG. 4 This is a photograph showing an ear of wheat in the example of test example 2.

FIG. 5: This photo shows the results of Test Example 2. On the left is the wheat in the example and on the right is the wheat in the comparison example.

FIG. 6: A photograph showing the results of Test Example 4. The corn planted in the right row is the example corn and the corn planted in the left row is the comparison corn.

FIG. 7: A photograph showing the results of Test Example 4. On the left is the corn in the example and on the right is the corn in the comparison example.

FIG. 8: A photograph showing the results of Test Example 8. On the left is a carrot from the comparison example and on the right is a carrot from the example.

FIG. 9: A photograph showing the results of Test Example 9. On the left is ginseng in the comparative example and on the right is ginseng in the example.

FIG. 10: A photograph showing the results of Test Example 11. On the left are leeks from the comparative example and on the right are leeks from the example.

FIG. 11: A photograph showing the results of Test Example 12. On the left is rice in the comparative example and on the right is rice in the example.

FIG. 12: A photograph showing the results of Test Example 13. On the left are fava beans in the comparative example, and on the right are fava beans in the example.

FIG. 13: This is a photograph showing the results of Test Example 14. On the right is the cabbage in the comparison example and on the left is the cabbage in the example.

### DESCRIPTION OF EMBODIMENTS

[Method of Production of the Extract]

The method of producing the extraction liquid includes a freezing step in which plant tissues are frozen.

Plant tissue for the freezing step can be obtained from any plant species. For example, Caricaceae, Bromeliaceae, Musaceae, Cucurbitaceae, Myrtaceae, Oxalidaceae, Moraceae, Malvaceae, Rubiaceae, Laureaceae, Passifloraceae, Sapindaceae, Clusiaceae, Ebenaceae, Rutaceae, Annonaceae, Arecaceae, Cactaceae, Rosaceae, Fabaceae, Poaceae can be used as examples.

More specifically, Carica, Ananas, Musa, Siraitia, Psidium, Averrhoa, Ficus, Theobroma, Coffea, Cinnamomum, Passiflora, Litchi, Garcinia, Diospyros, Casimiroa, Annona, Phoenix, Hylocereus, Cerasus, Glycine, Hordeum, Triticum, Zea can be used as an example.

Plant tissues for the freezing step are not limited, and examples include plant seeds, roots, shoots, stems, leaves, and petals. These tissues may be frozen as they are or partially excised and frozen in the form of sections when subjected to the freezing step.

In the freezing step, it is preferred to freeze the plant tissue in a state of being immersed in a liquid. As a liquid to immerse the plant tissue, a cryoprotective agent including an aqueous solution of dimethyl sulfoxide (DMSO), glycerin, ethylene glycol, saccharides, or the like is preferably used. Among them, it is preferred to use an aqueous saccharide solution, or in particular, an aqueous trehalose solution.

The upper limit of the lowest temperature during freezing in the freezing step is preferably −20° C. or less, more preferably −30° C. or less, furthermore preferably −40° C. or less, still more preferably −50° C. or less, and still furthermore preferably −55° C. or less.

Further, the lower limit of the lowest temperature during freezing is preferably −200° C. or more, more preferably −150° C. or more, furthermore preferably −100° C. or more, still more preferably −80° C. or more, still furthermore preferably −70° C. or more, and even still more preferably −65° C. or more.

In the freezing step, it is preferred to slowly decrease the temperature rather than rapidly decrease the temperature to the lowest temperature during freezing. From the viewpoint of the survival rate after thawing, the rate of temperature decrease is preferably 0.8° C./day or less, more preferably 0.6° C./day or less, furthermore preferably 0.5° C./day or less, still more preferably 0.3° C./day or less, still furthermore preferably 0.2° C./day, and even still more preferably 0.1° C./day.

In a case where the temperature is slowly decreased as described above, it is preferred to use a program freezer in the freezing step.

The lower limit of the period of the freezing step is preferably 100 days or more, more preferably 120 days or more, furthermore preferably 150 days or more, still more preferably 160 days or more, and still furthermore preferably 180 days or more.

In this regard, the expression “period of the freezing step” is referred to as a period of time from the time point when the temperature of a plant tissue is started to decrease until the time point when a thawing step is started.

The plant tissue that has undergone the freezing step may be used in the next extraction step in its frozen state, but it is preferably thawed before being used in the extraction step. The method of thawing is not particularly limited. The frozen plant tissue may be thawed naturally by leaving it at room temperature, or it may be thawed while rinsing the frozen plant tissue under running water. Preferably, natural thawing at room temperature is preferred.

It is preferable to include a selection step between the freezing step and the extraction step described below. The selection step is a process to select living plant tissues from the frozen plant tissues.

The specifics of the selection step are not limited as long as it is possible to select living plant tissue.

A preferred form of the selection step is a method that includes fermentation treatment of plant tissue that has undergone the freezing step. This is a method that takes advantage of differences in resistance to fermentation or other treatment by microorganisms between living and dead plant tissues.

Living plant tissue maintains its tangible state without being decomposed by microorganisms. On the other hand, dead plant tissue is decomposed by microorganisms and softens or liquefies. Therefore, after the fermentation treatment, living and dead plant tissues can be easily sorted.

The method of fermentation treatment is not limited. The method by which plant tissue that has undergone the freezing step is left in the open air is a good example.

In this case, the lower limit of the temperature of the environment to be left is preferably 0° C. or higher, more preferably 10° C. or higher, and even more preferably 20° C. or higher. The upper limit is preferably less than 50° C., more preferably less than 40° C.

The period of time for leaving the product in the open air is not limited as long as fermentation can take place, preferably from a few days to several weeks, specifically from 1 day to 4 weeks.

After the fermentation treatment, it is preferable to separate the dead plant tissue from the living plant tissue. The separation treatment is not limited to any specific form, as long as the living plant tissues can be separated from the mixture of dead and living plant tissues.

As mentioned above, after the fermentation treatment, dead plant tissue is decomposed by the microorganism and softened or liquefied. Therefore, dead plant tissue can be easily washed away and removed by washing plant tissue that has undergone the fermentation treatment. Washing with water is a good example of washing.

The present invention includes an extraction step to obtain an extraction liquid from plant tissue that has undergone a freezing step. The method of extraction is not particularly limited. The extractant used for extraction is preferably an aqueous solvent, more preferably water or an aqueous solution is an example.

The extractant should preferably contain sugars or sugar alcohols. More specifically, monosaccharides (glucose, lactose, threose, arabinose, xylose, galactose, ribose, glucose, sorbose, fructose, mannose), disaccharides (sucrose, lactose, maltose, trehalose, cellobiose, and Isomaltose, isotrehalose, isotrehalose, neotrehalose, neolactose, turanose, palatinose), other polysaccharides (trisaccharides: raffinose, melezitose, maltotriose, tetrasaccharides: acarbose, stachyose, glycogen, soluble starch, amylose, dextrin, and glucan, β1,3-glucan, fructan, N-acetylglucosamine, chitin, chitosan), sugar alcohols (xylitol, sorbitol erythritol, mannitol, maltitol), oligosaccharides (raffinose, panose, maltotriose, melezitose, gentianose, stachyose, cyclodextrin, xylo-oligosaccharides, cellulose oligosaccharides, lacto-oligosaccharides, fructooligosaccharides, galacto-oligosaccharides It is preferable to use an extractant containing one or more sugars or sugar alcohols selected from (mannan oligosaccharides).

More preferably, the sugar or sugar alcohol is an extraction liquid containing one or a combination of two types of sucralose and trehalose.

When an extractant that does not contain the above sugars or sugar alcohols is used, the above sugars or sugar alcohols may be added to the extraction liquid obtained after the extraction step.

It is preferable to perform a crushing treatment to crush the plant tissue that has undergone the freezing step, or more specifically, the living plant tissue that has undergone the freezing step. The crushing treatment is preferably exemplified by the crushing treatment.

The crushing treatment can be done with a mixer, ball mill, or other crusher, but crushing by grinding with a mortar is a preferred example.

The stress applied to the plant tissue in the crushing treatment is not limited, but it is preferable to crush it gently without applying too much stress. Particularly, a mortar and pestle are preferably used for gentle crushing.

The time spent in the crushing treatment is not limited, but is preferably from tens of seconds to several hours. Specifically, the lower limit is preferably 10 seconds or more, more preferably 30 seconds or more. The upper limit is preferably 10 hours or less, more preferably 5 hours or less, and even more preferably 3 hours or less.

The crushing treatment of plant tissue may be performed with the plant tissue immersed in the extractant, but preferably before the extractant is added. In other words, it is preferable to extraction liquid the plant tissue that has undergone the crushing treatment by bringing it into contact with the extractant.

Plant tissue that has undergone the freezing step is brought into contact with the extractant, or more specifically, the plant tissue is immersed in the extractant, and the components contained in the plant tissue are transferred to the extractant to obtain the extract.

After the extraction step, the extraction liquid may be filtered to remove plant tissue residue.

The amount of extractant used in the extraction step is not limited. The amount of extractant is preferably 0.5 parts by mass or more, more preferably 1 part by mass or more, more preferably 3 parts by mass or more, and even more preferably 5 or more parts by mass.

The upper limit is also not particularly limited, and the amount of extractant per 1 mass of plant tissue is preferably less than 100 parts by mass, more preferably less than 50 parts by mass The maximum amount is 20 parts by weight or less.

The immersion time in the extractant is not limited. The lower limit of immersion time is not limited, but can be preferably 1 minute or longer, more preferably 10 minutes or longer, and even more preferably 30 minutes or longer. The upper limit of soaking time is also not limited, but should preferably be less than 2 days, more preferably less than 1 day, preferably less than 12 hours, and even more preferably less than 6 hours.

The temperature of the extractant during immersion is also not limited. The lower limit is preferably 0° C. or higher, more preferably 10° C. or higher. The upper limit is preferably 60° C. or less, more preferably 50° C. or less, even more preferably 45° C. or less, even more preferably 40° C. or less.

If the crushing treatment yields a liquid or paste in which the components contained in the plant tissue are dissolved or dispersed, such liquid or paste is also included in the “extracted solution.

In this case, the crushing treatment itself constitutes the extraction step.

[Extraction Liquid and Extracted Dry Matter]

The extraction liquid is produced by the above production method.

The extracted and dried product obtained by drying the extraction liquid and removing the solvent is also included in the present invention. Methods of obtaining the extract dried product are not limited, but spray drying and lyophilization are examples.

In this Description, “extraction liquid” is not a term that refers only to the primary extraction liquid obtained through the extraction step. The term “extraction liquid” also includes dilution liquid in which the primary extraction liquid is diluted with any liquid, and concentration liquid in which the primary extraction liquid is concentrated. In addition, a solution obtained by dissolving the extract dried product in any solution, in other words, a solvent-exchanged solution of the primary obtained extract, is also included in “extraction liquid”. In addition, as mentioned above, when a liquid or paste in which components contained in plant tissues are dissolved or dispersed is obtained by the crushing process, such liquid or paste is also included in the “extraction liquid”.

The extracts and extract-dried products of the present invention can be used to enhance plant characteristics. In this description, “plant characteristics” is a concept that broadly includes without limitation environmental adaptation characteristics such as cold tolerance, high temperature adaptation, highland adaptation, lowland adaptation, growth rate, germination rate, growth uniformity, degree of rooting, fertility including quantity and size of fruits and seeds, sweetness, disease and insect resistance, drought resistance, and other plant characteristics.

The extracts and extract-dried products can be used to enhance one or more of the following plant characteristics: growth characteristics, cold tolerance, high temperature adaptation, germination rate, growth uniformity, degree of rooting, fertility, and drought tolerance.

The term “growth characteristics” is a concept that includes all characteristics of plant growth.

The term “Cold tolerance” refers to the ability of a plant to adapt to growth at temperatures lower than its natural optimum growth temperature.

The term “high temperature adaptation” refers to the ability of a plant to adapt to higher temperatures than its natural optimum growing temperature.

It is not limited to germination from seeds, but also includes germination from roots (root germination) and the percentage of germination from asexual propagules such as bulblets.

The term “growth uniformity” refers to the uniformity of the degree of growth of multiple plantlets treated under the same conditions.

The term “degree of rooting” refers to the degree of rooting in the soil.

The term “fertility” refers to the abundance of plant tissue (seeds, fruits, roots, leaves, or stems) harvested from a single individual plant.

The term “drought tolerance” refers to the tolerance to drought. It includes drought tolerance of plants growing in soil as well as of plants after they have been harvested from soil.

[Method for Enhancing Characteristics (1)

Plant tissue for the soaking step can be obtained from any plant species. For example, Caricaceae, Bromeliaceae, Musaceae, Cucurbitaceae, Myrtaceae, Oxalidaceae, Moraceae, Malvaceae, Rubiaceae, Laureaceae, Passifloraceae, Sapindaceae, Clusiaceae, Ebenaceae, Rutaceae, Annonaceae, Arecaceae, Cactaceae, Rosaceae, Fabaceae, Poaceae can be used as examples.

More specifically, Carica, Ananas, Musa, Siraitia, Psidium, Averrhoa, Ficus, Theobroma, Coffea, Cinnamomum, Passiflora, itchi, Garcinia, Diospyros, Casimiroa, Annona, Phoenix, Hylocereus, Cerasus, Glycine, Hordeum, Triticum and other plants can be used as examples.

The plant species of the plant tissue from which the extraction liquid used in the soaking step is derived and the plant species of the plant tissue used in the soaking step may be the same or different. In other words, plant tissues of plant species other than the specific plant species may be macerated in the extraction liquid obtained by freezing and extracting plant tissues of a specific plant species. Even in such interspecies application, the desired effect can be obtained according to the method for enhancing characteristics.

Plant tissues for the soaking step are not limited, and examples include plant seeds, roots, shoots, stems, leaves, petals, etc. These tissues may be soaked as they are when submitted to the soaking step, or they may be partially excised and soaked in the form of sections.

It is also preferable that the plant tissue whose characteristics are to be enhanced be dried prior to the soaking step. For example, the tissue may be sun-dried for a few days to dry the surface of the plant tissue. In addition, drying may be performed using a hairdryer or other dryer. This can increase the efficiency of penetration of the extraction solution into the plant tissue in the soaking step.

When seeds with seed coat are subjected to the soaking step, the thickness of the seed coat is preferably less than 3 cm, more preferably less than 1 cm, more Preferably 5 mm or less, more preferably 3 mm or less. If the thickness of the seed coat is within the above range, the penetration of the extractant is more efficient and the extractant can penetrate the seeds in a shorter time.

Grain seeds such as barley, wheat, soybeans, and rice are good examples of such seeds to which the invention is applied.

Soaking time is not limited, but preferably 30 minutes or more, preferably 1 hour or more, preferably 6 hours or more, preferably 1 2 hours or more, preferably 24 hours or more, even more preferably 48 hours or more, even more preferably 60 The time period should be at least 1.5 hours. The upper limit of soaking time is preferably less than 300 hours, more preferably less than 200 hours, and even more preferably less than 1 The time period can be set to 00 hours or less.

The temperature of the extraction liquid during the soaking step should be at a temperature that does not allow bacteria to grow during the same process. Preferably, the temperature should be at least 0° C., more preferably at least 5° C., and even more preferably at least 10° C. The upper limit is preferably less than 50° C., more preferably less than 45° C., even more preferably less than 40° C., even more preferably less than 35° C. or less, and even more preferably 30° C. or less.

The extractant used in the soaking step is preferably a dilute solution of the primary extraction liquid obtained by the extraction step, diluted with any liquid. By diluting the extraction liquid obtained primary by the extraction step, a large number of plant tissues can be used in the soaking step at one time, thereby improving production efficiency.

The dilution ratio is not limited. The volume of the diluted solution after dilution is preferably more than 100 times the volume of the plant tissue used for extraction, more preferably 1000 times or more, even more preferably 5000 times or more, even more preferably 8000. Even at such high dilution rates, the effects of the invention can be fully obtained.

Although the upper limit of the dilution ratio is not particularly limited, it is preferably less than 100,000 times, more preferably less than 50,000 times, and more preferably less than 20,000 times, more preferably 10,000 times.

When the liquid or paste obtained by crushing plant tissue without adding any extractant is diluted to obtain a dilute solution, the dilution ratio is preferably 100 times or more, more preferably 1000 times or more, even more preferably 5000 times or more, even more preferably It can be 8,000 times or more. Even at such high dilution ratio, the effects of the invention can be fully obtained.

In this case, the upper limit of the dilution ratio is not particularly limited, but preferably it should be less than 100000 times, more preferably less than 50,000 times, more preferably less than 20,000 times, more preferably less than 10,000 times.

The liquid used for dilution should be a liquid containing sugars or sugar alcohols as well as the extractant. More specifically, a liquid containing one or more sugars or sugar alcohols selected from Monosaccharides (glucose, Lactose, threose, arabinose, Xylose, Galactose, Ribose, Glucose, Sorbose, Fructose, Mannose), Disaccharides (Sucrose (sucrose), Lactose (lactose), Maltose (maltose), trehalose, Cellobiose, Isomaltose, Isotrehalose, Neotrehalose, Neolactose, Turanose, Palatinose), Other Polysaccharides (Trisaccharides: Raffinose, melezitose, maltotriose, Tetrasaccharides, Acarbose, Stachyose, Glycogen, Solubilized starch, Amylose, Dextrin, Glucan, Beta 1,3-glucan, fructan, N-acetylglucosamine, Chitin, Chitosan), Sugar alcohols (Xylitol, Sorbitol, Erythritol, Mannitol, Maltitol), Oligosaccharides (Raffinose, Panose, Maltotriose, Melezitose, Gentianose, Stachyose, Cyclodextrins, Xylooligosaccharides, Cellulose oligosaccharides, Lacto-oligosaccharides, Fructooligosaccharides, Galactooligosaccharides, Mannan oligosaccharides) should be used for dilution.

More preferably, a liquid containing one or a combination of two sugars or sugar alcohols, sucralose and trehalose, is used for dilution.

In the soaking step, the weight of plant tissue to be soaked per liter of extraction liquid is preferably 0.5 kg or more, more preferably 1 kg or more, more preferably 1.5 kg or more.

There is no maximum limit to the weight of plant tissue to be soaked per liter of extract, but preferably less than 3 kg, more preferably less than 2.5 kg.

In the soaking step, the entire plant tissue should be soaked in the extraction liquid. If the entire plant tissue is not soaked in the extraction liquid at one time, it may be achieved by rolling or stirring the plant tissue in the extraction liquid during the soaking step, so that the entire plant tissue is in contact with the extraction liquid.

Plant tissue subjected to the soaking step is enhanced with the same plant characteristics as when subjected to the freeze-thaw awakening method. The “plant characteristics” that can be enhanced by the method for enhancing characteristics of the present invention include without limitation the characteristics possessed by plants as described above.

Specifically, according to the method for enhancing plant characteristics, one or more of the following characteristics can be enhanced: environmental adaptation characteristics such as cold tolerance, high temperature adaptation, highland adaptation, lowland adaptation, growth rate, germination rate, growth uniformity, degree of rooting, fertility including quantity and size of fruits and seeds, sweetness, pest and disease resistance, and drought resistance.

The “germination rate” is the effect obtained when seeds are subjected to the soaking step.

By growing plant tissue with enhanced characteristics by the method of enhancing the characteristics of the present invention, plants with the enhanced characteristics can be obtained. The plant tissue after the soaking step can be sown as it is without any treatment.

The method of cultivation is not limited. If the plant tissue subjected to the soaking step is a seed of a plant, it can be sown according to the usual method to generate individual plants, which can then be cultivated according to the usual method.

If the plant tissue used in the soaking step is a plant part other than a seed, it can be transferred directly to soil or a culture medium and germinated, or it can be cut into small pieces and cultured in cell culture according to the usual method to generate individual plants by inducing callus, adventitious embryo, or adventitious bud.

The next generation of plants obtained by methods other than sexual reproduction from plants to which the method for enhancing characteristic of the present invention have been applied will inherit the enhanced characteristics. Therefore, if a plant with enhanced characteristics can be obtained by the method for enhancing characteristic of the present invention, the offspring of the next generation and beyond, derived from plant tissues other than seeds (e.g., cotyledons) that can generate plant individuals independent from the plant, will also have enhanced characteristics.

Plants subjected to the method of enhancing characteristics also exhibit enhanced characteristics even when used as scions for grafting.

For plants that bear fruits or seeds, the application of this method for enhancing characteristics can improve the size and yield of the fruits or seeds. Therefore, the merit of applying the present invention as a method of producing fruits or seeds is very significant.

In fruits or seeds produced in this way, sweetness and other nutritional components are enhanced, which is a very significant agro-industrial advantage.

[Method for Enhancing Characteristics (2)]

The method for enhancing the characteristics of plants can also be in a mode that comprises a spraying step in which the above-mentioned extraction liquid is sprayed on the plants for which the above-mentioned characteristics are to be enhanced.

The plant species of the plant tissue from which the extraction liquid used in the spraying step is derived and the plant species of the plant tissue to be used in the spraying step may be the same or different. In other words, the extraction liquid obtained by freezing and extracting the plant tissue of a particular plant species may be sprayed on plant species other than that particular plant species. Even in such interspecies application, the desired effect can be obtained according to the method for enhancing the characteristics.

In the spraying step, the state of the plants to which the extraction liquid is sprayed is not limited. The plants to be sprayed may be plants grown in soil such as fields, plants grown in potted plants or planters, or plants grown on hydroponic culture media.

The method of spraying the extraction liquid is not limited and can be done using a funnel or existing sprayer. The extraction liquid can also be sprayed on any part of the planted plant, e.g., buds, flowers, leaves, stems, tree branches, and soil (roots).

When the extraction liquid is applied to the above-ground portion of the plant, it may also be applied to the soil at the same time. Spraying the soil along with the above-ground portion of the plant allows the plant to absorb the extracted solution from the roots as well, and the property-enhancing effects of the extracted solution are further demonstrated.

The extraction liquid used in the spraying step should preferably be a diluted solution, in which the primary extraction liquid obtained by the extraction step is diluted with any liquid. By diluting the primary extraction liquid obtained by the extraction step, the extraction liquid can be sprayed on many plants.

The dilution ratio of the extraction liquid used in the spraying step is not particularly limited. The volume of the diluted solution after dilution can be preferably 100 times or more, more preferably 250 times or more, more preferably 2500 times or more, more preferably 12500 times or more, more preferably 2000 times or more, of the volume of the plant tissue used for extraction. The product can be used. Even at such high dilution ratio, the effects of the invention can be fully obtained.

The upper limit of the dilution ratio is not particularly limited, but preferably less than 1,000,000 times, and more preferably less than 500,000 times, more preferably less than 250,000 times, more preferably less than 125,000 times, more preferably less than 50,000 times, more preferably less than 25,000 times can be used as a guide.

The liquid or paste obtained by crushing plant tissue without adding any extractant can be diluted to obtain a dilute solution. In such cases, the dilution ratio is preferably 100 times or more, more preferably 250 times or more, and even more preferably 2,500 times or more, even more preferably 12,500 times or more, and even more preferably 20,000 times or more. Even at such high dilution rates, the effects of the invention can be fully obtained.

In this case, the upper limit of the dilution ratio is not particularly limited, but preferably less than 1,000,000 times, more preferably less than 500,000 times or less, and even more preferably 250,000 times or less, and even more preferably 125,000 times or less, more preferably 50,000 times or less, and even more preferably 25,000 times or less.

The amount of spray in the spraying process is not limited. For example, the standard amount of extraction liquid to be sprayed per 1 m2 of plant crop area is preferably 0.01 liter or more, more preferably 0.1 liter or more, more preferably 0.5 liter or more, and even more preferably 1 liter or more.

The standard amount of extract to be applied per 1 m2 of planted area is preferably 1000 liters or less. More preferably, 100 liters or less, and even more preferably, 10 liters or less.

The spraying step may be done only once during the growing season or multiple times during the growing season. If multiple applications are made during the growing season, they can be made, for example, every 1 day to 1 month, preferably every 2 days to 1 week.

The next generation of plants obtained by methods other than sexual reproduction from plants to which the method for enhancing the characteristics of the present invention has been applied will inherit the enhanced characteristics. Therefore, if a plant with enhanced characteristics can be obtained by the method of the present invention, the offspring of the next generation and beyond, which are derived from that plant, will also have the enhanced characteristics, as they are generated from plant tissues other than seeds (e.g., cotyledons) that can generate plant individuals independent of that plant.

Plants subjected to the method for enhancing characteristics also exhibit enhanced characteristics even when used as scions for grafting.

Other matters described in the above [Method for Enhancing Characteristics (1)] are directly applicable to the plant species to which this embodiment with a spraying step can be applied, the liquid used for dilution of the extraction liquid, and the characteristics of the plants to be enhanced.

In addition, this method may be implemented with both the soaking step and the spraying step described above.

[Method for Searching (1)

Plants treated with the method for enhancing characteristics of the present invention undergo significant changes in their gene expression profiles. It can be said that the increase or decrease in expression of a particular gene is the cause of the characteristic enhancement. In other words, genes whose expression levels increase or decrease in plant cells by application of the method for enhancing characteristics of the invention are genes involved in the enhancement of plant characteristics.

Therefore, it is possible to search for genes that enhance plant characteristics by analyzing and identifying genes whose expression increases or decreases in plant cells by applying the method for enhancing characteristics.

In other words, the invention also relates to a method for searching for genes involved in enhancing plant characteristics. The invention includes the steps of treating plants with the method for enhancing characteristics described above, and identifying genes whose expression levels were different in the treated plants compared to plants that have not undergone said treatment.

Specifically, the Method for Searching of the present invention includes the process of treating plants with the method for enhancing characteristics of the present invention described above, and the processes of (i) and/or (ii) below.

(i) Identifying genes that are highly expressed in the treated plants compared to non-treated plants.

(ii) identifying genes that are lower expressed in the treated plants compared to the non-treated plants.

Steps (i) and (ii) in the method for searching of the present invention can be carried out by the usual methods. For example, transcriptome analysis, such as microarray or RNA sequencing, can be used to identify genes with variable expression levels in plants treated with the method for enhancing the characteristics of the present invention.

[Method for Screening]

As mentioned above, since genes whose expression levels fluctuate in plants to which the method for enhancing characteristics is applied are factors that enhance characteristics, the expression levels of such genes can be used as indicators to screen for factors that enhance characteristics in plants.

In other words, the present invention also relates to a method for screening factors that enhance plant characteristics by using the variation in the expression levels of genes in plants to which the test substance is applied as an indicator. Here, “plants to which the test substance has been applied” includes plants to which the test substance has been introduced, plants treated with the test substance, plants in contact with or exposed to the test substance, etc.

Specifically, the following (i) and/or (ii) genes are used as indicators compared to plants that have not been treated by the above mentioned method for enhancing the characteristics of the present invention.

(i) Genes that are highly expressed in the treated plants.

(ii) Genes that show low expression levels in the treated plants.

When the expression level of the gene (i) in plants to which the test substance is applied is higher than the expression level of the gene in plants to which the test substance is not applied, the test substance is screened as a factor enhancing plant characteristics.

Also, when the expression level of the gene described in (ii) above in plants to which the test substance is applied is lower than the expression level of the gene in plants to which the test substance is not applied, the test substance is screened as a factor enhancing plant characteristics.

Gene expression levels can be confirmed by conventional methods such as Northern blotting and real-time PCR.

[Method for Analyzing Extraction Liquid]

Present invention also relates to a method for analyzing extraction liquid.

Specifically, the extraction liquid produced by the method of producing the extraction liquid described above is prepared for analysis. In addition, extraction liquid extracted from plant tissues that have not undergone the freezing step is prepared as a comparison object.

In order to achieve a highly accurate analysis, the manufacturing conditions of the extraction liquid of comparison object should match the manufacturing conditions of the extraction liquid of analysis object, except for the presence or absence of the freezing step.

Comparative analysis of the prepared extraction liquid of analysis object and the extraction liquid of comparison object is performed.

The method of comparative analysis can be performed using conventional methods, and mass spectrometer (LC-MS, GC MS-MS, etc.) are suitable examples.

Comparative analysis identifies components that are present in the extraction liquid of analysis object but not in the extraction liquid of comparison object, or components that are present in greater or lesser amounts in the extraction liquid of analysis object compared to the extraction liquid of comparison.

The method of identification is not limited. For example, when a mass spectrometer is used in the comparative analysis, the mass spectrum is compared and the component corresponding to the characteristic peak is identified. In this case, a database that can search for compounds based on m/z values may be used, or the component corresponding to the peak in question may be isolated and submitted to NMR measurement to identify the component. The components may be identified.

By conducting a comparative analysis of the components of the extraction liquid of analysis object and the extraction liquid of comparison object using the method for analysis of the present invention, it is possible to understand the changes brought to the plant tissue by the freezing step and, in turn, to clarify the details of the mechanism of action of the method for enhancing the characteristics of the present invention.

[Method for Searching (2)

The present invention also relates to a method for searching for factors for enhancing plant characteristics. Here, “factors for enhancing plant characteristics” are components contained in the extraction liquid of the present invention that can act on plant tissues, plant cells, or plants generated from them, and lead to enhancement of characteristics.

Specifically, the extraction liquid produced by the above-mentioned method for producing the extraction liquid of the present invention is prepared as the analysis object. In addition, extraction liquid extracted from plant tissues that have not undergone the freezing step is prepared as a comparative object.

In order to achieve highly accurate analysis, it is preferable that the manufacturing conditions of the extract solution to be compared match the manufacturing conditions of the extract solution to be analyzed, except for the presence or absence of the freezing step.

Comparative analysis of the prepared extraction liquid of analysis object and the extraction liquid of comparison object is performed.

The method of comparative analysis can be performed using conventional methods, and mass spectrometer (LC-MS, GC MS-MS, etc.) are suitable examples.

Comparative analysis identifies components that are present in the extraction liquid of analysis object but not in the extraction liquid of comparison object, or that are present in greater or lesser amounts in the extraction liquid of analysis object compared to the extraction liquid of comparison object.

Identification methods are not limited. For example, if a mass spectrometer is used in the comparative analysis, the mass spectra are compared and the component corresponding to the characteristic peak is identified. In this case, a database that can search for compounds based on m/z values may be used, or the component corresponding to the peak in question may be isolated and submitted to NMR measurement. The components may be identified.

In a preferred form of the invention, the process may further include an soaking step and a determination step. In the soaking step, plant tissue or plant cells are soaked in a solution containing one or more components identified by the identification step.

For the embodiment in which the plant tissue is soaked, the above description of the soaking step for the method for enhancing the characteristics of the present invention is directly applicable.

In the case of soaking plant cells, the plant cells may be seeded in a culture vessel and cultured in a culture solution containing the above-mentioned components.

In the determination step, if plant characteristics are enhanced in plant tissues or plant cells that have undergone the soaking step more than in plant tissues or plant cells that have not undergone the soaking step, the above components are determined to be factors that enhance plant characteristics.

A specific embodiment of the determination step is to observe plants generated from plant tissue or plant cells that have undergone the soaking step and plants generated from plant tissue or plant cells that have not undergone the soaking step.

Specifically, in the determination step, the characteristics of plants generated from plant tissues or plant cells that have undergone the soaking step are observed, and if an enhancement of the characteristics of the plants is observed compared to plants generated from plant tissues or plant cells that have not undergone the soaking step, the aforementioned component is determined to be a factor in enhancing the characteristics of the plants.

Plant characteristics to be observed here can be employed without limitation, including environmental adaptation characteristics such as cold tolerance, high temperature adaptation, highland adaptation, lowland adaptation, growth rate, germination rate, growth uniformity, degree of rooting, fertility including quantity and size of fruits and seeds, sweetness, pest and disease resistance, drought tolerance, and other characteristics that plants possess.

In the determination step, the determination may be performed by a genetic analysis method.

Specifically, the expression levels of the genes identified by the method described in the above section [Method for Searching (1)] are analyzed. When an increase in the expression level of the gene identified in (i) described in the same paragraph is observed, or when a decrease in the expression level of the gene identified in (ii) described in the same paragraph is observed, the above component is determined to be a factor for enhancing plant characteristics.

The increase or decrease in gene expression can be easily confirmed by Northern blotting or real-time PCR.

[Solutions for Enhancing Plant Characteristics, Methods for their Production and Methods for Enhancing Characteristics].

This invention also relates to solutions for enhancing plant characteristics and methods for their production.

Solutions for enhancing plant characteristics can be produced by adding to an aqueous medium the components that have been determined to be factors in enhancing plant characteristics by the inventive Method for Searching described above [Method for Searching (2)].

A suitable aqueous medium is the “liquid used for dilution” of the extraction liquid, as described in the above section [Method for enhancing characteristics].

The components to be added to the aqueous medium may be extracted from plant tissue. More preferably, the aforementioned components are obtained by isolation and purification from plant extracts.

The aforementioned components may also be obtained by artificial synthesis. The synthetic method may be any chemical or molecular biological method.

If the aforementioned component is a protein, it can be obtained by introducing an expression vector of the protein into an appropriate cell or bacterium, expressing the protein, and extracting it. When the aforementioned component is RNA, it can be chemically synthesized by an appropriate nucleic acid synthesis method according to conventional methods. When said component is polysaccharide, it can be chemically synthesized by hydrolysis-reverse reaction method, melting method, solvent method, etc. When said components are low molecular weight compounds, they can be synthesized by appropriate organic chemical synthesis methods.

By soaking the plant tissue in the solution produced in this way, the characteristics of the plant tissue can be enhanced. The description of the soaking step in the above [Method for Enhancing Plant Characteristics] is directly applicable to the form of the method for enhancing the characteristics of the plant tissue.

The solution produced by the method described above can be applied to plants whose characteristics are to be enhanced, thereby enhancing the characteristics of said plants. The description of the spraying step in the above [Method for enhancing characteristics] is directly applicable to the implementation of such a method for enhancing characteristics.

### EXAMPLE

### <Test Example 1> Enhancement of Wheat Characteristics Using Papaya Extract

Commercially available papaya seeds were frozen in a trehalose solution and placed in a program freezer. Freezing was performed slowly over 180 days at a temperature drop rate of 0.5° C./day, with a minimum temperature of −6° C. at freezing. The temperature was kept at 0° C.

Frozen papaya seeds were thawed naturally at room temperature (25° C.). They were left in the open air (25° C.) for one week. The dead seeds from the freezing step were fermented and softened or liquefied by exposure to the open air. The fermented seeds (i.e., dead seeds) were washed off by placing the seeds on a colander and rinsing them with water, and only the living seeds were sorted.

The remaining, live seeds in the colander were gently grinded and crushed using a mortar and pestle to obtain a paste. 1 cc of the paste was diluted to 10 L with an aqueous sucralose and trehalose solution (about 8 The diluted extraction liquid was prepared by diluting the extraction liquid from 8000 to 10,000 times.

20 kg of wheat “Fukuhonoka” seeds were soaked in 10 L of diluted extraction liquid and The product was left in place for 72 hours. The soaked seeds were allowed to dry naturally in the sun for a few days before soaking.

Seeds that had undergone this soaking step were sown on Sep. 30, 2019 in a field in Kamiarihan, Takahashi, Okayama, Japan. As a comparison, untreated seeds of the same variety were also sown. Note that the growing area is located in a cold region where the temperature is below freezing in December.

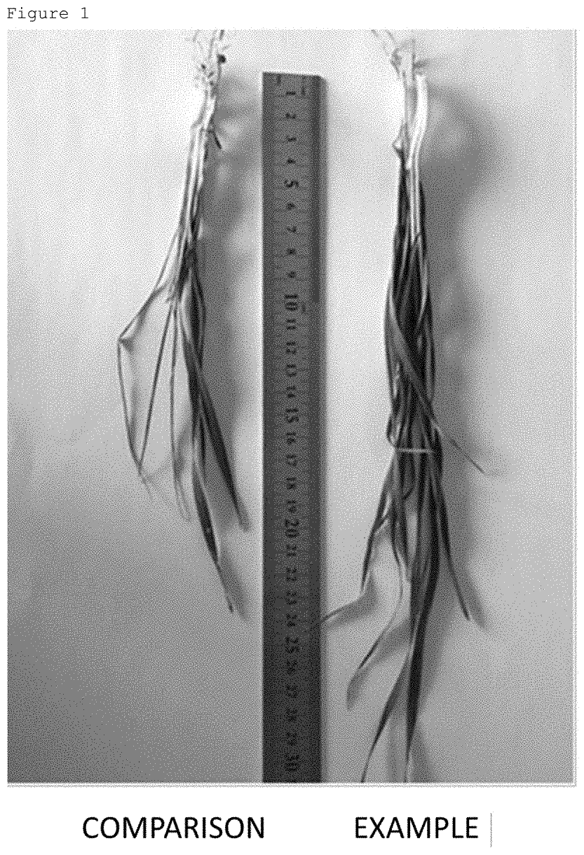

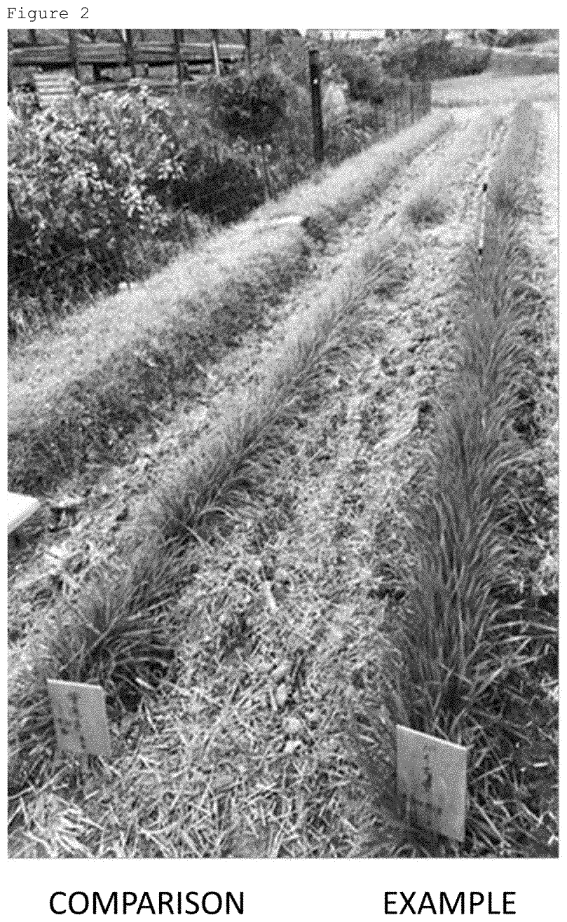

FIG. 1 shows the comparative and example wheat as of Nov. 2, 2019. Photographs are shown. FIG. 2 also shows a photo of the Nov. 13, 2019 Photographs of the wheat cultivation in the comparative and example.

As shown in FIGS. 1 and 2, the growth rate of the example wheat is significantly higher than that of the comparative example wheat. The wheat in the example sprouted within 3 to 4 days after sowing, the growth rate was uniform, and there was no sparse growth compared to the wheat in the comparative example. The wheat in the example also had firm roots, and the green color of the leaves was darker than in the comparison wheat. The amount of chlorophyll is probably different. This faster growth rate also makes it more difficult for weeds to grow, which may have the advantage of eliminating the need for herbicides. In addition, given that the growing area is in a severely cold region, the wheat in the example also has improved cold tolerance (see FIG. 2).

Furthermore, the germination rate of wheat seeds in the example was found to be higher than that of wheat seeds in the comparison example. The yield of wheat in the example was also higher than that of the comparative example.

The wheat in the examples and the comparative example that was pulled from the soil was left in the open air for a while. After leaving the wheat to stand, the wheat in the case of implementation was clearly more moist to the touch than the wheat in the case of comparison. In other words, the wheat in the example was clearly more moist than the wheat in the comparison example (see FIG. 1).

These results indicate that soaking of plant tissue in the extraction liquid extracted from frozen plant tissue can enhance the growth characteristics and cold tolerance, as well as the germination rate, growth uniformity, degree of rooting, fertility, and drought tolerance of the immersed plant tissue and the plants that develop from the tissue.

### <Test Example 2> Enhancement of Wheat Characteristics Using Papaya Extract

Wheat seeds soaked in papaya seed extraction liquid were obtained using the same procedure as in Test Example 1. These were sown and cultivated in November 2019 at a farm in Miyazaki, Miyazaki Prefecture, Japan. As a comparison, untreated seeds of the same variety were also sown.

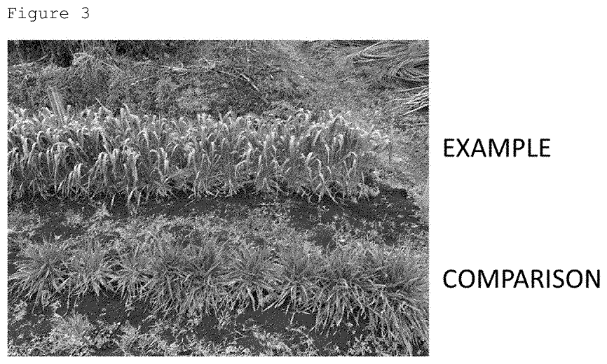

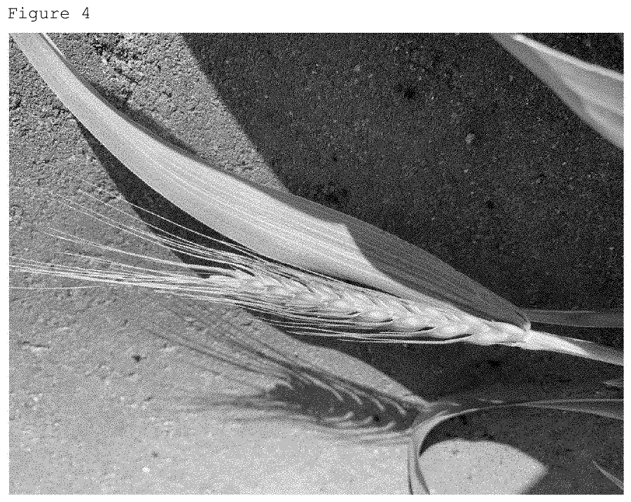

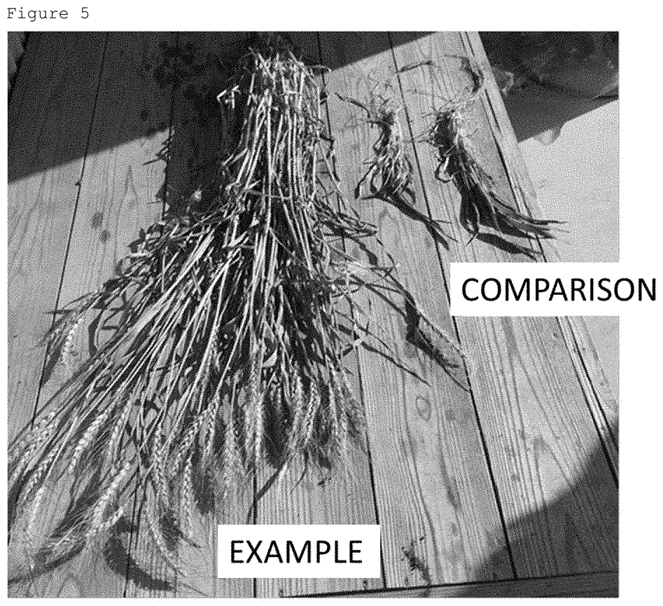

FIG. 3 shows the wheat in the case and comparison cases at the end of January 2020 (about 90 days later). The following are photographs. FIG. 4 shows a photograph of an ear of wheat in the example taken on the same day. FIG. 5 shows a photograph of the wheat from the example and comparison cases taken on Mar. 5, 2020.

A significant increase in growth rate (about three times faster than the comparative case) was observed in the example wheat (see FIG. 3), and fruiting was observed despite the cold weather at the end of January (see FIG. 4). As in test example 1, an improvement in the uniformity of the growth rate (see FIG. 3) and an enhancement of degree of rooting were observed. In addition, the wheat in the example also showed improved cold tolerance, as it grew and bore fruit at a remarkable rate despite being grown in winter from November to January (see FIG. 3 and FIG. 5). (See 4). In addition, early in the morning, there was a large amount of frost on the wheat in the comparison case, but no frost at all on the wheat in the case study. Frost is a cause of plant death, and measures such as frost protection are usually necessary, but the example wheat did not need frost protection, and it is recognized as very suitable for cultivation in cold regions.

In addition, when the wheat was collected on March 5, the comparative case on the right had no ears at all, while the case on the left had grown more than three times as long and had many ears (see FIG. 5). Surprisingly, these were each grown from a single seed, and the example had more stems and more ears than the comparative example. These results may suggest that two-season cropping of wheat is possible. In both cases, no fertilizer was applied at all.

Furthermore, the germination rate of wheat seeds in the example was found to be higher than that of wheat seeds in the comparative example. In addition, as in Test Example 1, an improvement in drought resistance was also observed.

These results indicate that soaking of plant tissue in the extraction liquid extracted from frozen plant tissue can enhance the growth characteristics and cold tolerance, as well as the germination rate, growth uniformity, degree of rooting, fertility, and drought tolerance of the immersed plant tissue and the plants that develop from the tissue. [02]<Test Example 3> Enhancement of wheat characteristics using wheat extract

The same procedure was used as in Test Example 1, except that wheat seeds were used instead of papaya seeds in the preparation of the extract. That is, the extraction liquid was prepared from wheat seeds that had undergone the freezing step, wheat seeds were soaked in the extract, and these seeds were sown in the field for cultivation.

The results showed that, similar to the results of test examples 1 and 2, treated wheat showed significantly improved growth characteristics and cold tolerance, as well as germination rate, growth uniformity, degree of rooting, fertility, and drought tolerance compared to untreated wheat.

The results of Test Examples 1-3 indicate that the plant species of the plant tissue used in the soaking step may be the same or different from the plant species from which the extraction liquid used in the soaking step is derived. In other words, this invention can be applied to both homologous and heterologous species.

### <Test Example 4> Enhancing Corn Characteristics Using Papaya Extract

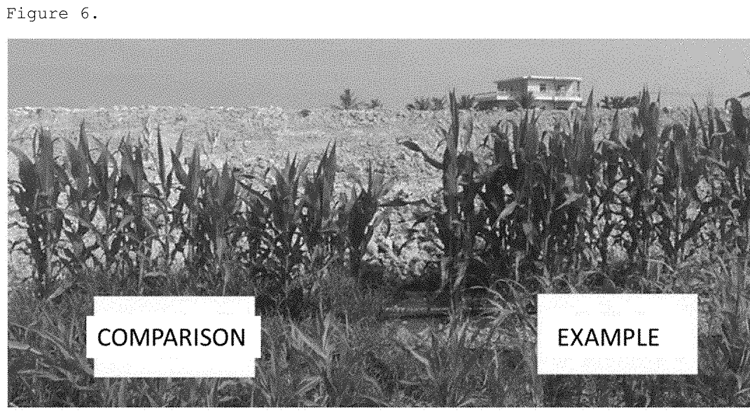

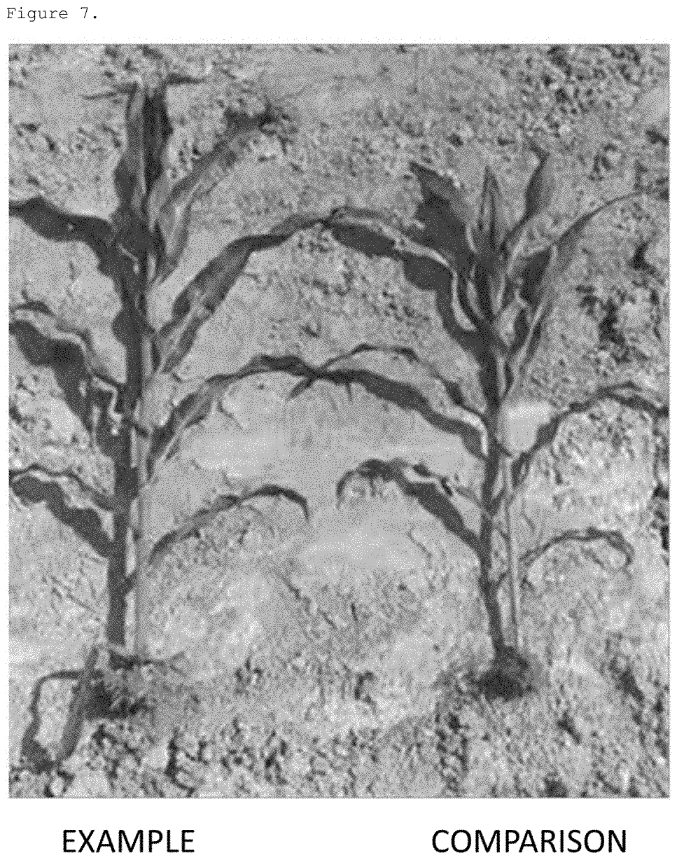

An extraction liquid was prepared from papaya seeds by the same method as in Test Example 1, and corn seeds were soaked in said extraction liquid by the same method as in Test Example 1. These corn seeds were grown in Hainan Province (Hainan Base) in China at the end of 2019 (November (sown on the 29th) showed a marked improvement in growth characteristics and cold tolerance compared to growing untreated corn seeds (see FIGS. 6 and 7). As with the wheat, the color was darker and the roots were more firmly established. In addition, the treated corn yielded about four times more than the untreated corn. Although the growing area is also prone to typhoons, the roots grew well and did not fall over in the wind, and this is believed to be one of reason for the better yield.

Furthermore, the corn in the example showed a uniform growth rate (FIG. 6). In addition, the corn seeds in the example showed a higher germination rate than the seeds in the comparative example.

### <Test Example 5> Enhancing Soybean Characteristics Using Papaya Extract

An extract was prepared based When these soybean seeds were grown in a cold region of China, a significant improvement in growth characteristics and cold tolerance was observed compared to growing untreated soybean seeds. As in test examples 1-4, improvements in germination rate, growth uniformity, degree of rooting, fertility, and drought resistance were also observed.

### <Test Example 6> Enhancement of Wheat Characteristics Using Papaya Extract

An extract was prepared from papaya seeds by the same method as in Test Example 1, and wheat seeds were soaked in said extract by the same method as in Test Example 1. When the wheat seeds were grown in the cold region of China, the growth characteristics and cold tolerance of the wheat seeds were significantly improved compared to those of the untreated wheat seeds.

As in test example 1, enhancement of germination rate, growth uniformity, degree of rooting, fertility, and drought resistance was also observed.

### <Test Example 7> Enhancement of Wheat Characteristics Using Papaya Extract

An extract was prepared from papaya seeds by the same method as in Test Example 1, and wheat seeds were soaked in said extract by the same method as in Test Example 1. The wheat seeds were sown in permafrost in Russia and harvested only two months after sowing. The yield was 13 tons per hectare, four times the normal yield. Because the cultivation area is permafrost, weeds cannot take root. In addition, because of their fast growth rate, they absorb all the nutrients from the soil. Therefore, the use of herbicides was not necessary for cultivation. When the ground was dug up to observe the condition of the wheat roots in the example, they were able to root into the completely frozen frozen soil. Although the amount of precipitation during the period when Test Example 7 was conducted was not sufficient to provide the amount of water necessary for wheat cultivation, it is assumed that the wheat in the example was growing by absorbing water from the ice contained in the frozen soil. In addition, it is thought that if the remaining leaves and stems are included after the fruit is harvested and the land is tilled together, nutrients can be returned to the soil, thus allowing for agriculture with less soil load. As in test example 1, enhancement of germination rate, growth uniformity, and drought resistance was also observed.

These results indicate that the application of the invention can significantly improve plant growth characteristics, cold tolerance, fertility, germination rate, growth uniformity, rooting, and drought tolerance.

### <Test Example 8> Enhancement of Carrot Characteristics Using Wheat Extract

Wheat seeds were soaked in a trehalose solution and then frozen in a program freezer. Freezing was performed slowly over 180 days at a temperature drop rate of 0.5° C./day, with a minimum temperature of −60° C. at freezing. The temperature was kept at 0° C.

Frozen wheat seeds were thawed naturally at room temperature (25° C.). They were left in the open air (25° C.) for one week. The dead seeds from the freezing process were fermented and softened or liquefied by exposure to the open air. The seeds were placed on a colander and rinsed with water to wash off these fermented seeds (i.e., dead seeds), and only the living seeds were sorted.

The remaining, living seeds in the colander were gently grinded and crushed using a mortar and pestle to obtain a paste. The diluted extract was prepared by diluting 1 cc of the paste to 10 L with an aqueous solution of sucralose and trehalose (diluted approximately 8000 to 1000 times).

Carrot seeds were soaked in the diluted extract solution for 72 hours. The seedlings were sown in soil in Kakamigahara City, Gifu Prefecture, on March 21, and cultivation began. As a comparative example, carrot seeds that had not been treated with the invention were sown and cultivated under the same values and conditions as in the case study.

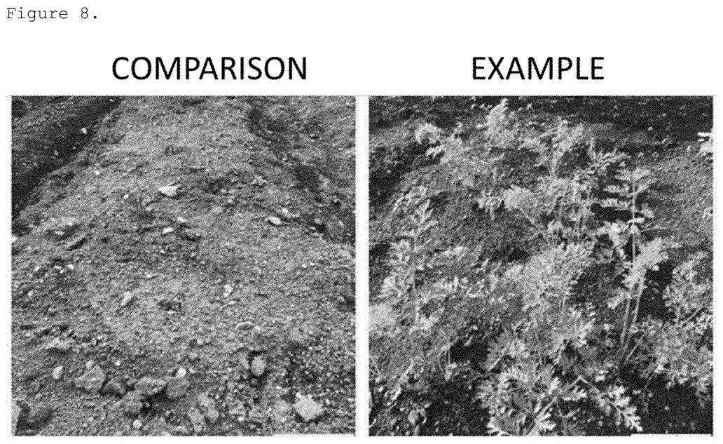

FIG. 8 shows a photograph of the cultivation of carrots in the case study and comparison at 38 days after the start of soil cultivation. As shown in the figure, many of the seeds in the comparative example did not germinate, while most of the seeds in the example germinated. Generally, carrot seeds are known to be difficult to germinate. The carrot seeds to which the invention was applied showed an improvement in germination rate. In addition, in terms of the growth speed of the germinated seedlings, while the comparative example was slow, the carrot in the example was extremely fast and could be harvested earlier than the comparative example. Furthermore, it is known that carrots prefer a relatively cool climate and are susceptible to high temperatures during the period of straight root growth. In the example, about 4 cm of straight root enlargement was already observed at the time 38 days after the start of soil cultivation (see FIG. 8), which allowed harvesting during the high temperature period of summer.

As mentioned above, carrots subjected to the invention showed improved germination rate, growth acceleration, and high temperature acclimation. These results indicate that the invention is highly effective in inducing germination when applied to crops with difficult germination rates. And even crops that prefer cooler climates showed enhanced growth characteristics as a result of enhanced high temperature adaptation.

### <Test Example 9> Enhancing the Properties of Asian Ginseng Using Wheat Extract

Extracts of wheat seeds were obtained by the same procedure as in Test Example 8. Asian ginseng roots were macerated in the diluted extract solution and left for 72 hours. The roots after this soaking process were planted in the soil of a field in Kibichuo-cho, Kaga-gun, Okayama Prefecture, on May 11, 2020, and cultivated The project was initiated. As a comparative example, Asian ginseng roots that had not been treated with the invention were simultaneously grown in the same location under the same conditions as in the case study.

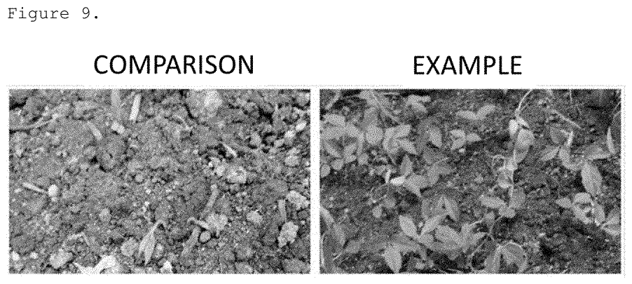

FIG. 9 shows the results after 42 days (6 weeks) from the start of soil cultivation, 2020. Photographs of Asian ginseng cultivation as of June 22 are shown for the case and comparison cases. As shown in the figure, many of the Asian ginseng plants in the comparative case did not germinate from the roots, and even if they did germinate, their growth rate was slow.

On the other hand, the Asian ginseng in the conducted case germinated from all the cultivated roots, and its growth rate was extremely fast, several times faster than that of the comparative case.

Furthermore, Asian ginseng is known to prefer a relatively cool climate and is susceptible to high temperatures during the period of straight root growth. In the example, direct root enlargement was already confirmed at 42 days after the start of soil cultivation (see FIG. 9), allowing harvesting during the high-temperature summer season.

As mentioned above, Asian ginseng plants subjected to the invention showed improved germination rate, growth acceleration, and high temperature adaptation. These results indicate a high germination induction effect when applied to crops with difficult germination rates. And even crops that prefer cooler climates showed enhanced growth characteristics as a result of enhanced high temperature adaptation.

### <Example 10> Other Plants

Seeds of the plants listed below were treated with an extraction liquid from papaya seeds using the same method as in Test Example 1, sown and grown. The cultivation was conducted in Okayama Prefecture, Japan.

Coffee, chili hazelnut, golden eggfruit, banana, dwarf coconut, cacao, lychee, palm palm, prickly pear, durian, cashew, carob, pawpaw mango, acacia, cypress, pineapple, guava, acai, dates, bakpari, danieli

The results showed that in all of the plant species listed above, treatment with the extraction liquid enhanced growth characteristics and cold tolerance, as well as germination rate, growth uniformity, degree of rooting, fertility, and drought tolerance.

These results indicate that the method for enhancing characteristics of present invention is effective for all plant species.

### <Test Example 11> Characteristic Enhancement by Spraying (Green Onion)

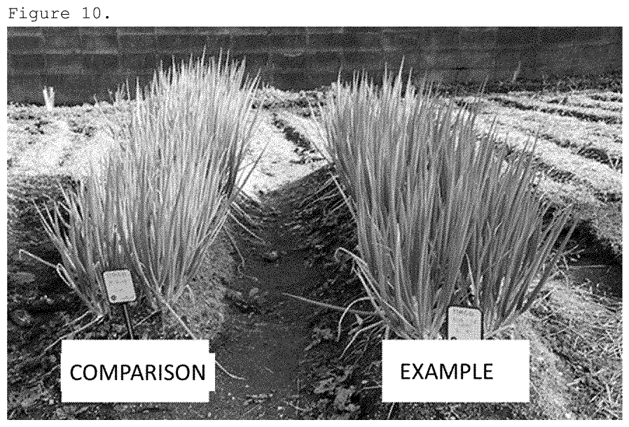

An extraction liquid was prepared from wheat seeds that had undergone the freezing step in the same manner as in Test Example 3. About 50 ml of this extraction liquid per shoot was sprayed on green onions two weeks after planting, every five days. As a comparative example, leeks grown without spraying of the extraction liquid were also prepared. FIG. 10 shows a photograph of the green onions at about 3 months (November) after planting. As shown in FIG. 10, a marked increase in growth rate was observed in the green onions grown after spraying with the extraction liquid. The green onions in the cases of implementation and comparison were harvested and weighed per shoot and the size of the above-ground portion was measured. As a result, the green onion in the example was observed to have a significantly improved yield compared to the green onion in the comparison example.

### <Test Example 12> Characteristic Enhancement by Spraying (Rice)

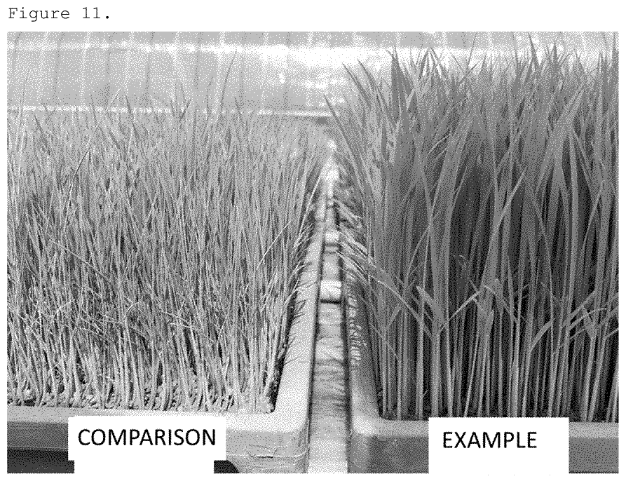

An extract was prepared from wheat seeds that had undergone the freezing process in the same manner as in Test Example 3. The extraction liquid was sprayed on rice seedlings and cultivated. As a result, a significant increase in growth rate was observed compared to rice grown without the application of the extraction liquid (FIG. 11). When the rice seedlings were planted and cultivated in rice paddies, a significant enhancement in growth rate, cold tolerance, and yield was observed compared to the rice seedlings cultivated in the comparative example.

### <Test Example 13> Characteristic Enhancement by Spraying (Fava Beans)

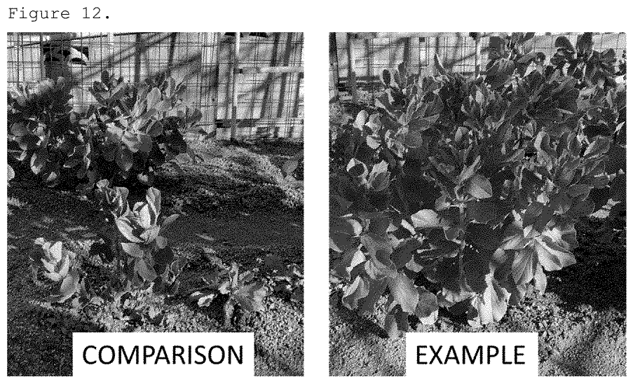

An extraction liquid was prepared from wheat seeds that had undergone the freezing step in the same manner as in Test Example 3. This extraction liquid was sprayed on soil-grown fava beans in Kure City, Hiroshima Prefecture, Japan. As a result, the fava beans in the example showed a remarkable increase in growth rate compared to those in the comparison case grown under the same conditions without spraying the extraction liquid (FIG. 12). The photo shown in FIG. 12 was taken on December 31. It can be said that the fava beans in the example showing significant growth in the middle of winter have acquired remarkable cold tolerance.

### <Test Example 14> Characteristic Enhancement by Spraying (Cabbage)

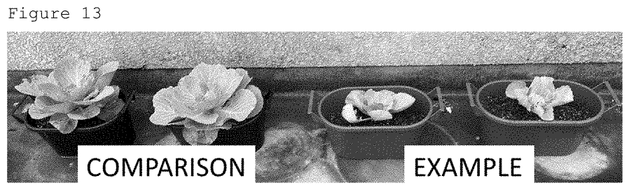

An extraction liquid was prepared from wheat seeds that had undergone the freezing step in the same manner as in Test Example 3. This extraction liquid was sprayed on cabbage grown in a planter. As the cabbage continued to grow, it was confirmed that it grew at a remarkable rate compared to the cabbage in the comparison case grown under the same conditions without the extraction liquid sprayed (FIG. 13). The photo shown in FIG. 13 was taken in Okayama Prefecture in January. The cabbage in the example that shows remarkable growth in the middle of winter can be said to have acquired remarkable cold tolerance.

### <Test Example 15> Characteristic Enhancement by Spraying (Other Plants)

Extracted from wheat seeds by the same method as in Test Example 3, this extraction liquid was sprayed while growing tomatoes, green peppers, wheat, morning glories, and watermelons. As a result, it was confirmed that growth characteristics, cold tolerance, growth uniformity, rooting, fertility, and drought tolerance were enhanced in all the plants.

### <Consideration>

The application of a method that undergoes a freezing step to freeze plant tissues, i.e., the freeze-thaw-awakening method (Ref. 3), enables the application of the following plant characteristics, specifically, environmental adaptation characteristics such as growth rate, cold tolerance, high temperature adaptation, highland and lowland adaptation characteristics, fruit and seed quantity and size, sweetness, pest and disease resistance, and drought tolerance, etc. can be enhanced. The plant species whose characteristics can be enhanced are not limited, and all plant species tested to date, such as Papaya, pineapple, banana, coffee, lohan fruit, guava, star fruit, fig, cacao, Ceylon cinnamon, passion fruit, lychee, mangosteen, black sapote, white sapote, thorn-leaf sugar apple, date palm, red dragon fruit, almond, soybean, and Wheat, barley, corn, have been shown to produce the desired effects. It is clear from the plant species listed here that the freeze-thaw awakening method is not a technology that can only be applied to certain plant strains, but is a universal technology that can be applied across plant species.

It is said that 97% of plant genes are asleep. Plants treated by the freeze-thaw awakening method with enhanced characteristics are then subjected to De novo RNA-seq was used for expression analysis. The results have recently shown that the expression levels of thousands of genes are altered in the treated group compared to the untreated group.

As a result of the analysis, increased expression of genes related to growth, such as plant hormones, and genes related to various environmental stress responses, such as salt, high temperature, low temperature, and drought, are observed.

The fact that the characteristics enhanced by the freeze-thaw awakening method are inherited without loss through growth or strain division indicates that the gene expression profiles described above are not lost through cell division.

Given the observed changes and maintenance of gene expression profiles as described above, it is clear that epigenetic changes are involved in the enhancement of characteristics by the freeze-thaw awakening method.

In other words, it can be understood that the freezing step triggers the activation of transcription of the gene in the region encoding the gene that leads to the enhancement of the plant characteristics described above, by the addition of an epigenetic marker that positively regulates the expression of the gene. Conversely, in the regions encoding genes that control the plant characteristics described above, transcription of the genes is repressed by the addition of epigenetic markers that repress the expression of the genes.

The results of the test examples herein are discussed based on this finding. As shown in test examples 1-11, the same property enhancement was observed in plant tissue soaked in the extraction liquid of plant tissue that underwent the freezing step as in the case of the application of the freeze-thaw awakening method. Also, as shown in test examples 12-15, in plants grown by spraying the extracts of plant tissues that have undergone the freezing step, the same property enhancements have been observed as when subjected to the application of the freeze-thaw awakening method. In other words, it can be understood that the same epigenetic gene expression profile changes occur in plant tissues subjected to the invention as in plant tissues subjected to the freeze-thaw awakening method.

In fact, several plant species to which this method for enhancing characteristics was applied were used as samples for De novo expression analysis was performed by RNA-seq. Similar variations in gene expression profiles were observed between plants subjected to the freeze-thaw awakening method and those subjected to the freeze-thaw awakening method.

The common denominator between the present invention and the freeze-thaw awakening method is the freezing process. In light of this similarity, it can be reasonably concluded that the freezing step triggers some specific factor that induces epigenetic changes and the resulting enhancement of characteristics.

In the freeze-thaw awakening method, it is conceivable that the above-mentioned specific factor generated by the freezing step acts on the plant cells, thereby inducing epigenetic changes.

On the other hand, in the present invention, it is understood that the specific factor generated by the freezing step is contained in the extraction liquid, and the specific factor acts on the cells constituting the plant tissue soaked in the extraction liquid, thereby inducing epigenetic changes in the plant cells as in the freezing and thawing awakening method.

In conclusion, the present invention is considered to have the same property-enhancing effect as the freeze-thaw awakening method through the specific factors mentioned above. In other words, it can be understood that the plant characteristics that can be enhanced by the freeze-thaw awakening method can also be enhanced by the application of the present invention.

In light of the fact that the freeze-thaw awakening method can be applied to all plant species without limitation and that the effects of the present invention were confirmed in various plant species in Test Examples 1-15, we believe that the present invention, like the freeze-thaw awakening method, can be applied to all plant species. It can be understood that this is not a technology that can only be applied to a specific strain of plants, but is a universal technology that can be applied across plant species.

Furthermore, Test Examples 1, 2, 4-15, which demonstrated that enhancement of characteristics is possible even when papaya- or wheat-derived extraction liquid is applied to a large number of plant species that are phylogenetically distant from each other. The results are noteworthy. It is known that plant gene sequences, protein amino acid sequences, and plant hormones are highly homologous even among phylogenetically distant heterologous species. The results of these test cases prove the broad applicability of the invention based on this high degree of homology between different plant species.

In other words, it can be reasonably understood that the aforementioned specific factor is not only compatible with a certain plant species, but is a factor with high heteroscedasticity that is compatible with a wide range of plants in general. Therefore, it can be understood that the desired effect can be obtained in the present invention no matter what combination of plant species from which the extract is derived and the plant species used in the soaking step.

In addition, in test examples 1-7, 10, and 12-15, the cold resistance enhancement effect was observed. On the other hand, in Test Examples 8 and 9, the effect of enhancing high-temperature adaptation was observed. This result suggests that the present invention does not selectively enhance either cold tolerance or high temperature adaptability, but rather extends the appropriate temperature range for plant growth (improvement of growth temperature adaptability). This is supported by the results of gene expression analysis of plants adapted to the invention.

### INDUSTRIAL APPLICABILITY

The invention is applicable to crop production technology.

1. A method for producing an extraction liquid, comprising
a freezing step for freezing plant tissue and
an extraction step for obtaining an extraction liquid from the plant tissue that has undergone the freezing step.

2. The method for producing an extraction liquid according to claim 1, wherein the extraction liquid for enhancing the characteristics of a plant.

3. The method for producing an extraction liquid according to claim 2, wherein the characteristics of a plant are one or more of the following:
plant growth characteristics, cold tolerance, high temperature adaptation, germination rate, growth uniformity, degree of rooting, fertility, and drought tolerance.

4. The method for producing an extraction liquid according to claim 1, wherein comprising a selection step for selecting living plant tissue from the frozen plant tissue, and
the selection step is performed between the freezing step and extraction step.

5. The method for producing an extraction liquid according to claim 1, wherein in the freezing step, the plant tissue is frozen to −20° C. or lower over 100 days while lowering the temperature at a rate of 0.8° C./day or less.

6. The method for producing an extraction liquid according to claim 1, wherein the plant tissue is frozen while immersed in a sugar solution in the freezing step.

7. (canceled)

8. The method for producing an extraction liquid according to claim 4, wherein in the selection step, a fermentation treatment is performed on the plant tissue that has undergone the freezing step.

9. (canceled)

10. (canceled)

11. (canceled)

12. (canceled)

13. (canceled)

14. The method for producing an extraction liquid according to claim 1, wherein in the extraction step, a living plant tissue is subjected to a crushing treatment.

15. (canceled)

16. (canceled)

17. The extraction liquid produced by the producing method according to claim 1.

18. The extraction liquid according to claim 17, wherein the extraction liquid is for enhancing plant characteristics.

19. The extraction liquid according to claim 18, wherein the extraction liquid contains sugars or sugar alcohols.

20. (canceled)

21. (canceled)

22. The extract dried product produced by drying the extraction liquid according to claim 17.

23. A method of enhancing characteristics of a plant tissue, comprising:
a soaking step in which plant tissue from which the characteristics of the plant are to be enhanced is soaked in the extraction liquid,
wherein said extraction liquid is
the extraction liquid according to claim 17.

24. (canceled)

25. The method of enhancing characteristics of a plant tissue according to claim 23, wherein a soaking time in the soaking step is from 1 to 100 hours.

26. A method for producing plant tissue with enhanced plant characteristics comprising applying the method of enhancing characteristics of a plant tissue according to claim 23.

27. A plant tissue with enhanced plant characteristics produced by applying the method according to claim 26.

28. A method for producing a plant with enhanced plant characteristics, comprising the step of growing the plant tissue according to claim 27.

29. A method for enhancing plant characteristics, comprising a spraying step for spraying the extraction liquid on the plants for which the plant characteristics are to be enhanced,
wherein said extraction liquid is
the extraction liquid according to claim 17.

30. A method for producing a plant with enhanced plant characteristics, comprising applying the method for enhancing plant characteristics according to claim 29.

31. A plant with enhanced plant characteristics produced by the method for producing according to claim 28.

32. The plant according to claim 31, wherein the plant characteristics are one or more of the following:
plant growth characteristics, cold tolerance, high temperature adaptation, germination rate, growth uniformity, degree of rooting, fertility, and drought tolerance.

33. A plant tissue obtained from the plant according to claim 32, and used as a scion for grafting.

34. (canceled)

35. A method for producing fruits or seeds of said plant comprising cultivating the plant according to claim 32.

36. Fruits or seeds produced by the method according to claim 35.

37. A method for searching for genes involved in enhancing plant characteristics, comprising;
A step of treating plants by the method according to claim 23, and
(i) a step for identifying genes that are highly expressed in the treated plants as compared to plants that have not received said treatment, and/or
(ii) a step for identifying genes that are expressed at lower levels in the treated plants as compared to plants that have not received said treatment.

38. (canceled)

39. A method for analyzing the extraction liquid comprising
preparing an extraction liquid produced by the method according to claim 1 as an analysis object,
preparing, as a comparison object, an extract solution extracted from plant tissue that has not undergone the freezing process described above.
comparing and analyzing the extraction liquid of the analysis object and the extraction liquid of the comparison object, and
identifying a component wherein the component is
a component contained in the extraction liquid of the analysis object, but not in the extraction liquid of the comparison object and/or
a component contained more or less in the extraction liquid of the analysis object compared to the extraction liquid of the comparison object.

40. (canceled)

41. (canceled)

42. (canceled)

43. (canceled)

44. (canceled)

45. (canceled)

46. (canceled)

47. (canceled)

48. (canceled)

49. A method of enhancing characteristics of a plant tissue, comprising:
a soaking step in which plant tissue from which the characteristics of the plant are to be enhanced is soaked in the extraction liquid,
wherein said extraction liquid is
the extraction liquid produced by dissolving the extract dried product according to claim 22.

50. A method for enhancing plant characteristics, comprising a spraying step for spraying the extraction liquid on the plants for which the plant characteristics are to be enhanced,
wherein said extraction liquid is
the extraction liquid produced by dissolving the extract dried product according to claim 22.

51. A method for searching for genes involved in enhancing plant characteristics, comprising;
A step of treating plants by the method according to claim 29, and
(i) a step for identifying genes that are highly expressed in the treated plants as compared to plants that have not received said treatment, and/or
(ii) a step for identifying genes that are expressed at lower levels in the treated plants as compared to plants that have not received said treatment.
